(19) United States
(12) Patent Application Publication
(10) Pub. No.: US 2021/0399320 A1
(43) Pub. Date: Dec. 23, 2021

(54) DETERMINING THE MASS FLOW OF AN EXHAUST GAS CONSTITUENT OF A FUEL CELL

(71) Applicant: AVL LIST GMBH, Graz (AT)

(72) Inventors: Gerbert Orasche, Graz (AT); Tomas Dehne, Nestelbach (AT); Roland Wanker, Hart bei Graz (AT)

(21) Appl. No.: 17/292,814

(22) PCT Filed: Nov. 12, 2019
(86) PCT No.: PCT/AT2019/060383

§ 371 Date: May 11, 2021

(30) Foreign Application Priority Data
Nov. 12, 2018 (AT) A50971/2018

(51) Int. Cl. H01M 8/0438; H01M 8/0444; H01M 8/04492; G01N 33/00; G01N 21/3504; G01F 1/66

(52) U.S. Cl. CPC H01M 8/04402; H01M 8/0441; H01M 8/04462; H01M 8/0447; G01F 1/661; H01M 8/04522; G01N 33/004; G01N 21/3504; H01M 8/04514

(57) ABSTRACT

Various embodiments of the present application are directed to methods of measuring a mass flow rate of at least one exhaust gas constituent in an exhaust gas of a fuel cell. In one example embodiment, the method includes the steps of measuring a volumetric flow rate of the exhaust gas; using a gas sensor to determine a concentration of the at least one exhaust gas constituent, and calculating the mass flow rate of the exhaust gas constituent using the volumetric flow rate of the exhaust gas and the determine concentration of the at least one exhaust gas constituent.

The present invention relates to a method for measuring the mass flow rate of at least one exhaust gas constituent in the exhaust gas of a fuel cell.

For the operation of a fuel cell, the mass flow rate of the operating media on the anode side and the cathode side (usually fuel and oxygen) is an important variable. Conventional fuel cells use hydrogen H₂ on the anode side and oxygen O₂ (usually in the form of air) on the cathode side as operating media for the electrochemical reaction. The hydrogen can be supplied in various ways, either as pure hydrogen, such as in a proton exchange membrane fuel cell (PEMFC), or in a chemical compound, such as in a phosphoric acid fuel cell (PAFC) or direct methanol fuel cell (DMFC). For the operation of the fuel cell, it is important to regulate the supplied gas flows precisely, since incorrect gas amounts or incorrectly conditioned (in particular pressure, temperature, humidity) gases, in particular on the cathode side, can lead to the fuel cell being damaged, or even destroyed. This largely applies to every type of fuel cell.

It is often also desirable to determine the mass of an exhaust gas flow of the fuel cell, for example for a mass balance of the fuel cell between the supplied and removed mass. The sum of the supplied oxygen O₂, or air, and fuel, for example hydrogen Hz, should correspond to the mass flow rate of the exhaust gas. The mass balance is therefore also an important indicator of the operation of a fuel cell. For example, leaks in the fuel cell (more supplied mass than removed mass) can be detected.

A variation in the addition of hydrogen H₂ on the inlet side during operation of the fuel cell causes changes in the water content in the exhaust gas. Due to the changing water content in the exhaust gas, however, a constant molar mass of the exhaust gas cannot be assumed, which makes it difficult to determine the mass of the exhaust gas flow since the water content in the exhaust gas is of course not known. This means that an exhaust gas mass flow rate cannot be easily derived from the volume flow rate of the exhaust gas. However, the humidity of the exhaust gas could be measured by means of humidity sensors and thus the water content in the exhaust gas could be inferred, which would allow the exhaust gas mass flow rate to be determined. However, humidity sensors are rather imprecise and are also not able to follow rapid changes in the water content, as can occur when a fuel cell is in operation.

Likewise, undesired chemical reactions in the fuel cell can be inferred on the basis of the analysis of the exhaust gas flow of a fuel cell. One example of this is what is referred to as carbon corrosion of a PEMFC. In particular operating states, in particular when the fuel cell is switched on and off, the carbon of the electrode on the cathode side reacts with water or the oxygen O₂ to form carbon dioxide CO₂ and/or carbon monoxide CO. However, the concentrations of carbon dioxide CO₂ and/or carbon monoxide CO in the exhaust gas are so low that the masses of these substances can hardly be measured. The resulting CO₂ and/or CO is removed together with the exhaust gas. Carbon corrosion damages the fuel cell over its operating life, which reduces the efficiency and, in the case of lasting damage, can also lead to failure of the fuel cell. As is known, carbon corrosion occurs in particular during start/stop processes, as studies have shown, for example in A. B. Ofstad, et al., “Carbon Corrosion of PEMFC During Shut-down/Start-up when using an Air Purge Procedure,” ECS Transactions, 16 (2) 1301-1311 (2008). In order to detect carbon corrosion, the concentration of CO and CO₂ in the exhaust gas is measured using a non-dispersive infrared sensor (NDIR).

The composition of the exhaust gas flow of a fuel cell is therefore an interesting variable for the operation of a fuel cell, both for a fuel cell on a test stand for development or further development, as well as in the use of the fuel cell itself, for example in a vehicle. The detection of carbon corrosion is particularly important for development on the test stand in order to determine the acceptable operating parameters or to check and further develop the structure of the fuel cell or operating strategies themselves. For this purpose, in particular the concentration of carbon dioxide and/or carbon monoxide in the exhaust gas, as a measure of damage to the fuel cell, is an interesting characteristic variable for the operation of the fuel cell on the test stand. However, measuring the composition of the exhaust gas flow of a fuel cell is very difficult. This is because these substances are only contained in very small amounts in the exhaust gas, and also because particular exhaust gas constituents are not or only insufficiently accessible for direct measurement.

DE 100 48 183 B4 discloses a device for monitoring a hydrogen-containing gas flow. A small flow is diverted from a laminar flow line to a container and returned again to the laminar flow line from the container. A specific sensor for measuring the hydrogen mass flow rate is arranged in the container. The sensor has a membrane arrangement which, according to the reaction of hydrogen with an oxidizing agent, generates an electric current that is proportional to the hydrogen mass flow rate in the removed flow. A flow sensor arranged in the laminar flow line is used to determine the flow in the laminar flow line. Due to the laminar flow, the hydrogen mass flow rate in the laminar flow line can be calculated using the measured flow and the proportionality between the flows.

It is therefore an object of the present invention to provide a method and an arrangement in order to be able to measure the mass flow rate of an exhaust gas constituent of the exhaust gas of a fuel cell simply and reliably.

This object is achieved according to the invention in that the volume flow rate of the exhaust gas is measured, and a gas sensor is used to determine the concentration of the at least one exhaust gas constituent, and volume flow rate of the exhaust gas and the determined concentration are used to calculate the mass flow rate of the exhaust gas constituent. In this way, the mass flow rate can be determined very precisely because the volume flow rate can be determined without being influenced by exhaust gas constituents in the exhaust gas.

For this purpose, a non-dispersive infrared sensor is preferably used as the gas sensor. To determine the volume flow rate, the speed of the exhaust gas in an exhaust gas line is preferably detected and, from this, the volume flow rate is determined using the known cross-sectional area of the exhaust gas line, since the speed can be simply detected without being influenced by exhaust gas constituents.

Preferably, the gas sensor is used to determine the concentration of water in the exhaust gas, and the mass flow rate of the water, as the exhaust gas constituent, is determined, because water is an important exhaust gas constituent and an important indicator of the operation of the fuel cell.

In order to determine the total exhaust gas mass flow rate, for example for a mass balance, a known mass flow rate of a main constituent (either the cathode gas or the anode gas) of the exhaust gas and the mass flow rate of the water are used to determine an exhaust gas mass flow rate of the exhaust gas. For this purpose, the mass flow rate of the main constituent of the exhaust gas can be determined simply using the known density of the main constituent of the exhaust gas and the volume flow rate.

In addition, the gas sensor can be used to determine the concentration of carbon dioxide and/or carbon monoxide in the exhaust gas, which are also important indicators of the operation of the fuel cell, in particular as an indication of harmful carbon corrosion in the fuel cell.

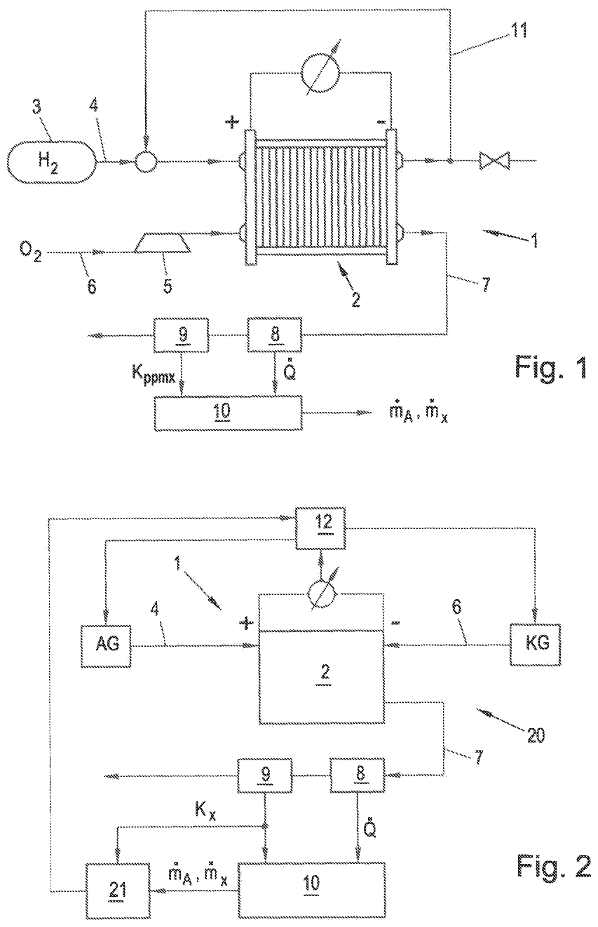

The present invention will be described in more detail in the following with reference to FIGS. 1 and 2, which schematically show, in a non-limiting manner, advantageous embodiments of the invention by way of example. In the drawings:

FIG. 1 shows a fuel cell with determination of the exhaust gas mass flow rate of an exhaust gas constituent and the concentration of an exhaust gas constituent; and

FIG. 2 shows a test stand for a fuel cell for developing the fuel cell.

FIG. 1 shows a typical arrangement of a fuel cell 1 comprising a fuel cell stack 2 having a number of cells, in which stack the electrochemical reaction takes place in a known manner. The invention is described using the example of a PEMFC as the fuel cell 1, although other types of fuel cells are of course also conceivable. On the anode side, hydrogen Hz, for example from a hydrogen tank 3, is supplied as fuel via a suitable supply line 4. The excess hydrogen H₂ can be removed or returned to the supply step via a return line 11. On the cathode side, oxygen O₂, also in the form of air, is supplied via a suitable supply line 6. For this purpose, a fan 5 for conveying the oxygen O₂ can also be provided in the supply line 6 for the oxygen O₂. The exhaust gas of the fuel cell 1, in this case on the cathode side, is removed via an exhaust gas line 7. The relevant operating medium of the fuel cell 1, in this case for example the cathode gas (air, oxygen), is contained in the exhaust gas as a main constituent, but also contains water H₂O as a further essential exhaust gas constituent and possibly other gaseous exhaust gas constituents that result from operation of the fuel cell 1, for example CO or CO₂ in the event of carbon corrosion. One problem here is that neither the water content nor the amount of gaseous exhaust gas constituents is known. The water content, as the exhaust gas constituent, changes dynamically during operation, very rapid changes being entirely possible. This means that the water content in the exhaust gas, and thus also its specific weight, is not constant. The same largely also applies to the amount of gaseous exhaust gas constituents. Consequently, the concentration of such exhaust gas constituents, and thus the resulting mass or the exhaust gas mass flow rate \dot{m}_{A} of the exhaust gas, is not known.

In order to be able to nevertheless determine the exhaust gas mass flow rate \dot{m}_{A} of the exhaust gas, a volume flow rate sensor 8 is arranged in the exhaust gas line 7, which sensor is used to determine the volume flow rate \dot{Q} of the exhaust gas. A gas sensor 9 is also arranged in the exhaust gas line 7, which sensor is used to determine the concentration K (for example in ppm (parts per million)) of at least one exhaust gas constituent of the exhaust gas, for example water H₂O and/or CO₂ and/or CO. The order in which the volume flow rate sensor 8 and the gas sensor 9 are arranged in the exhaust gas line 7 does not matter per se. In the described embodiment having a PEMFC as the fuel cell 1, the exhaust gas is examined on the cathode side. However, there may be other types of fuel cells where the exhaust gas is examined on the anode side.

As the volume flow rate sensor 8, a well-known pitot flow sensor is used, for example, which determines the speed of the exhaust gas (for example in m/s) in the exhaust gas line 7. A pitot flow sensor is impervious to water in the exhaust gas, which is why such a sensor is particularly advantageous in this application. The known cross-sectional area (for example in m²) of the exhaust gas line 7 can then be used to easily calculate the volume flow rate \dot{Q} (for example in m³/s). This calculation can take place in the volume flow rate sensor 8 or only in the evaluation unit 10, in the latter case the flow speed of the exhaust gas being transferred from the volume flow rate sensor 8 to the evaluation unit 10, in which the cross-sectional area is known. Of course, other suitable volume flow rate sensors 8 can also be used, which sensors can be used to determine the volume flow rate \dot{Q} without being influenced by exhaust gas constituents, in particular water.

An optical sensor, such as a sensor that operates on the principle of spectroscopy, for example a non-dispersive infrared sensor (NDIR), is used as the gas sensor 9, for example. Of course, other suitable gas sensors 9 can also be used. The gas sensor 9 measures the concentration Kₚₚₘₓ of the exhaust gas constituent x, for example in ppm, based on the volume.

The mass flow rate \dot{m}ₓ (e.g., in g/s) of the exhaust gas constituent x can then be determined in the evaluation unit 10 from the concentration Kₚₚₘₓ (in ppm) and the volume flow rate \dot{Q} (e.g., in m³/s).

As is known, the molar mass mₘₒₗₓ (of the exhaust gas constituent x and the molar volume Vₘₒₗ can be used to calculate the concentration Kₓ in g/m³ of the exhaust gas constituent x according to the relationship

$${K}_{x} = \frac{{K}_{p p m x} \cdot {m}_{m o l x}}{{V}_{mol} \cdot {10}^{6}} .$$

The molar volume under normal conditions (273.15° K, 101325 Pa) is 22.414 liters, i.e., 22.414·10⁻³ m³. For example, the molar mass of carbon dioxide CO₂ is 44.01 g/mol, of carbon monoxide CO is 28.01 g/mol and of water is 18.01528 g/mol. If that is multiplied by the volume flow rate \dot{Q}, this results in the mass flow rate \dot{m}ₓ (e.g., in g/s) of the relevant exhaust gas constituent x, in particular for water H₂O, carbon dioxide CO₂ and/or carbon monoxide CO.

The mass flow rate \dot{m}ₓ determined in this way can then be used, for example, for a mass balance. In normal operation, the amount (e.g., in g/s) of the substances supplied has to correspond to the amount (e.g., in g/s) of the removed substances. The amount of the operating media supplied to the fuel cell 1, for example hydrogen H₂ and oxygen O₂ (air), can be assumed to be known because these are typically set by a controller of the fuel cell 1 and can be provided thereby. These amounts can also optionally be measured before entering the fuel cell 1. The basic composition of the exhaust gas of the fuel cell 1 is of course also known and consists of a main constituent and at least one exhaust gas constituent. For a PEMFC, the exhaust gas on the cathode side consists largely of oxygen O₂ (air) as the main constituent and water H₂O as the essential exhaust gas constituent. Since the volume flow rate \dot{Q} is known from the measurement using the volume flow rate sensor 8, the mass flow rate of the main constituent of the exhaust gas can be inferred, for example, from the known density of the main constituent (oxygen O₂ (air)). The determined mass flow rate \dot{m}ₓ of the at least one exhaust gas constituent x is added to this, and the total exhaust gas mass flow rate \dot{m}_{A} of the exhaust gas is obtained. This means that the mass balance can be continuously checked.

Of course, other exhaust gas constituents can also be taken into account for a mass balance. However, since, for example, the concentration of CO and CO₂ in a PEMFC is usually very low, these can also be disregarded for a sufficiently precise mass balance of a PEMFC fuel cell 1.

The mass balance can then be used, for example, to detect a leak in the fuel cell 1. For this purpose, at least the concentration K_{ppmH2O} of water H₂O, as the exhaust gas constituent x, is determined. If the exhaust gas mass flow rate \dot{m}_{A} determined thereby is less than the sum of the supplied fuel and oxygen per unit of time (it being also possible for a particular tolerance band to be defined), a leak can be inferred. This can be used both on a test stand 20 for the fuel cell 1 or also during normal use of the fuel cell 1, for example in a vehicle, in order to identify possible fault conditions. For this purpose, the evaluation unit 10 can also be integrated in a control unit of the fuel cell 1.

If the gas sensor 9 is used to determine the concentration K_{ppmCO2} (in ppm) or K_{CO2} (in g/m³) of carbon dioxide CO₂ and/or the concentration K_{ppmCO} (in ppm) or K_{CO} (in g/m³) of carbon monoxide CO, then this concentration can also be used independently of the determination of the exhaust gas mass flow rate \dot{m}_{A} or of a mass flow rate \dot{m}ₓ of an exhaust gas constituent x. The presence of carbon dioxide CO₂ and/or carbon monoxide in the exhaust gas indicates harmful carbon corrosion. If the concentration K_{ppmCO2} of carbon dioxide CO₂ and/or the concentration K_{ppmCO} of carbon monoxide CO in the exhaust gas is detected over time, progressive damage to the fuel cell stack 2 can be inferred. This can also be used both on a test stand for the fuel cell 1 or also in use, for example in a vehicle. The concentration K_{ppmCO2} of carbon dioxide CO₂ and/or the concentration K_{ppmCO} of carbon monoxide CO in the exhaust gas, or the sum over time, can also be output as a value, for example as a measure of damage.

A test stand 20 for a fuel cell 1 is shown in FIG. 2. The fuel cell 1 is set up on the test stand 20 and is operated on the test stand 20. The fuel cell 1 is controlled by means of a fuel cell control unit 12, for example by controlling the cathode gas KG and anode gas AG, e.g., the mass flow rate, the temperature, the pressure, the relative humidity, etc. For this purpose, the fuel cell control unit 12 can also process an output variable for the fuel cell 1, for example an electric voltage or an electric current, as indicated in FIG. 2. The fuel cell control unit 12 can also receive control commands from an external means, for example from a test stand control unit 21, in order to control the fuel cell 1. The test stand control unit 21 can process or evaluate a determined exhaust gas mass flow rate \dot{m}_{A} and/or a mass flow rate \dot{m}ₓ of an exhaust gas constituent x and/or a concentration Kₓ, Kₚₚₘₓ of an exhaust gas constituent x, in particular for the development of the fuel cell 1, which can also include the development of operating strategies, for example how the operating media are controlled. For this purpose, the evaluation unit 10 can also be integrated in the test stand control unit 21.

The concentration K_{CO2}, K_{ppmCO2} of carbon dioxide and/or carbon monoxide K_{CO}, K_{ppmCO}, as a measure of damage to the fuel cell, is preferably determined on the test stand 20 under particular operating conditions of the fuel cell 1, and used for the development of the fuel cell 1. In this way, switch-on and switch-off procedures of the fuel cell 1 can be particularly advantageously optimized as a specific operating strategy, since carbon corrosion is known to occur in particular during start/stop processes. This can be carried out on the test stand 20 under reproducible conditions. The determined concentration Kₓ, Kₚₚₘₓ of an exhaust gas constituent x and/or a mass balance and/or determined mass flow rates can also be used on the test stand 20 to control the test stand 20, and to that effect also to control the execution of a test run. In this way, for example, an emergency shutdown of the test stand 20 can be implemented in order to prevent permanent damage to or even destruction of the fuel cell 1. On the test stand 20, as a result of the test run carried out, it is quite possible that the fuel cell 1 is operated in an impermissible operating range, which can thereby be brought under control. For this purpose, for example, limit values for particular concentrations Kₓ, Kₚₚₘₓ and/or mass flow rates \dot{m}ₓ of an exhaust gas constituent x and/or a mass balance could be monitored.

1. A method for measuring a mass flow rate of at least one exhaust gas constituent in an exhaust gas of a fuel cell, the method including the steps of:
measuring a volumetric flow rate of the exhaust gas, and
using a gas sensor to determine a concentration of the at least one exhaust gas constituent, and
calculating the mass flow rate of the exhaust gas constituent using the volumetric flow rate of the exhaust gas and the determined concentration of the at least one exhaust gas constituent.

2. The method according to claim 1, wherein the gas sensor is a non-dispersive infrared sensor.

3. The method according to claim 1, further including detecting a speed of the exhaust gas in an exhaust gas line; and wherein the step of measuring a volumetric flow rate of the exhaust gas uses a known cross-sectional area of the exhaust gas line and the detected speed of the exhaust gas in the exhaust has line.

4. The method according to claim 1, wherein the exhaust gas constituent is water; and
wherein the gas sensor is configured and arranged to determine the concentration of water in the exhaust gas.

5. The method according to claim 4, further including determining an exhaust gas mass flow rate based upon a known mass flow rate of a main constituent of the exhaust gas and the mass flow rate of the water.

6. The method according to claim 5, wherein the step of calculating the mass flow rate of a main constituent of the exhaust gas utilizes a known density of the main constituent of the exhaust gas and the measured volumetric flow rate of the exhaust gas.

7. The method according to claim 5, further including the step of using the determined exhaust gas mass flow rate for a mass balance between an amount of substances supplied to the fuel cell and substances within the exhaust gas.

8. The method according to claim 1, wherein the gas sensor is further used to determine a concentration of carbon dioxide and/or the concentration of carbon monoxide in the exhaust gas.

9. The method according to claim 8, further including the step of determining an indication of carbon corrosion in the fuel cell based upon the concentration of carbon dioxide and/or the concentration (of carbon monoxide in the exhaust gas.

10. The method according to claim 2, wherein the exhaust gas constituent is water; and
the gas sensor is configured and arranged to determine a concentration of the water in the exhaust gas.

11. The method according to claim 3, wherein the exhaust gas constituent is water; and
the gas sensor is configured and arranged to determine a concentration of the water in the exhaust gas.

12. The method according to claim 6, further including the step of using the determined exhaust gas mass flow rate for a mass balance between an amount of substances supplied to the fuel cell and substances removed within the exhaust gas.

13. The method according to claim 2, wherein the gas sensor is further used to determine a concentration of carbon dioxide and/or the concentration of carbon monoxide in the exhaust gas.

14. The method according to claim 3, wherein the gas sensor is further used to determine a concentration of carbon dioxide and/or the concentration of carbon monoxide in the exhaust gas.
